(19) United States
(12) Patent Application Publication
(10) Pub. No.: US 2009/0312626 A1
(43) Pub. Date: Dec. 17, 2009

(54) ARTICLES AND METHODS FOR IMPROVING MRI IMAGING

(76) Inventors: Christopher J. Hanrahan, Salt Lake City, UT (US); B. J. Manaster, Salt Lake City, UT (US); Scott E. Jenack, Salt Lake City, UT (US); Jason N. Lance, Salt Lake City, UT (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/481,986

(22) Filed: Jun. 10, 2009

Related U.S. Application Data
(60) Provisional application No. 61/060,826, filed on Jun. 12, 2008.

(51) Int. Cl. A61B 5/055; A41D 19/00; A43B 17/00; A41D 27/00

(52) U.S. Cl. 600/421; 2/161.7; 2/239; 2/243.1

(57) ABSTRACT

Described herein are articles and methods for improving MRI imaging. The articles and methods augment fat saturation, which improves the resolution of the MRI image and ultimately provides more precise and accurate diagnosis of diseases.

### CROSS REFERENCE TO RELATED APPLICATIONS

This application claims priority upon U.S. provisional application Ser. No. 61/060,826, filed Jun. 12, 2008. This application is hereby incorporated by reference in its entirety.

### BACKGROUND

Magnetic resonance imaging (MRI) is a critical tool for the evaluation of bone infection, or osteomyelitis, localized soft tissue infection (i.e., cellulitis), and concentrated infected fluid collections (i.e., abscesses). While radiographs and bone scans have been used somewhat successfully in the past, MRI is increasingly recognized as the modality of choice.

Foot-related osteomyelitis is the primary or secondary reason for admission in approximately 75,000 hospitalizations each year, of which diabetes is responsible for the majority. Prompt diagnosis of cellulitis or osteomyelitis versus frank abscess formation is particularly important as it determines whether antibiotics alone are adequate or whether a surgical procedure may be necessary for debridement or amputation. The imaging diagnostic tools for evaluating foot infection have improved over the last decades and MRI has emerged as the most useful tool for early diagnosis. Surgical literature support the use of MRI as it prevents unnecessary surgery in diabetic patients who have difficulty with surgical recovery due to their disease.

Unfortunately, MRI of the foot and toes is not without technical problems. For accurate diagnosis, fat saturation MRI sequences are necessary to obtain acceptable sensitivity and specificity for the diagnosis of soft tissue viability and the presence of osteomyelitis. In the absence of fat saturation, enhanced MRI images had a sensitivity and specificity of 79 and 53 percent, respectively, whereas in the presence of fat saturation the sensitivity and specificity increased to 88 and 93 percent, respectively. When evaluating the feet, hands and elbows where air/tissue interfaces are in close proximity to the anatomy, artifacts produced by the air/tissue interface can obscure tissue detail, rendering accurate diagnosis difficult. While the literature does not address the degree to which fat saturation artifacts masks diagnosis of osteomyelitis or renders the MRI uninterruptible, efforts have been made to increase fat saturation. For example, obscuring the tissue/air interface has been attempted by placing water bags over the region of interest during MRI examination. Results are mixed, with some improvement in fat saturation, but fat saturation is often incomplete. Thus, what is needed are articles and methods for increasing fat saturation and ultimately improving MRI imaging of a body part that is generally difficult to image.

### SUMMARY

Described herein are articles and methods for improving MRI imaging. The articles and methods augment fat saturation, which improves the resolution of the MRI image and ultimately provides more precise and accurate diagnosis of diseases associated with, for example, diabetes. The advantages of the invention will be set forth in part in the description which follows, and in part will be obvious from the description, or may be learned by practice of the aspects below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 shows the use of a viscoelastic polymer to increase fat saturation and improve MRI imaging of a foot.

FIGS. 2-4 demonstrate the improved accuracy of osteomyelitis diagnosis using a viscoelastopolymer to decrease fat saturation artifacts in a diabetic patient with osteomyelitis of the big toe and the absence of osteomyelitis involving adjacent toes.

### DETAILED DESCRIPTION

Before the present methods and/or articles of manufacture are disclosed and described, it is to be understood that the aspects described below are not limited to specific articles, manufacture, or methods as such may of course vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a viscoelastic polymer” includes mixtures of two or more such polymers.

“Optional” or “optionally” means that the subsequently described event or circumstance can or cannot occur, and that the description includes instances where the event or circumstance occurs and instances where it does not. For example, the phrase “the article optionally contains laces” means that the article may or may not have laces.

Described herein are methods for improving MRI imaging of a body part. The first step of the method involves placing an article made of a viscoelastic polymer on the surface of a body part of a patient that is to be imaged. While the methods are not specific to any specific body part, the article should be placed on a section of the body in a manner such that it is in intimate contact with the body part undergoing MRI. After the article is placed on the body part, the body part is subsequently imaged by MRI. The methods described herein provide a convenient and efficient way to improve accurate visualization of body parts by MRI.

In one aspect, the method improves imaging by increasing the fat saturation of the body part that is being imaged. It is common during MRI to produce artifacts that make diagnosis of certain conditions less precise and less accurate. State of the art imaging images a body part without any material or with saline/water bags to improve fat saturation. The methods disclosed herein improve or enhance fat saturation of a body part during an MRI.

Placing the article in intimate contact with the body reduces air pockets, which create the artifacts. Thus, the phrase “intimate contact” as used herein refers to positioning or placing the article on the body part of the subject to be imaged such that there is minimal to no air present between the article and the body part. For example, the article can be placed directly on the body part. Not wishing to be bound by theory, by reducing the amount of air at the interface between the article and the body part, fewer artifacts are produced during imaging resulting in better visualization. The improved visualization can lead to more accurate diagnosis and in some cases the improved imaging can lead to a different treatment plan. For example, the methods and articles described herein are useful in imaging feet. The foot is very sensitive to air pockets and thus artifacts are commonly observed when imaged.

The article used in the methods described herein is made of a viscoelastic polymer. “Viscoelasticity” is the property of materials that exhibit both viscous and elastic characteristics when undergoing deformation. Viscous materials resist shear flow and strain linearly with time when a stress is applied. Elastic materials strain instantaneously when stretched and just as quickly return to their original state once the stress is removed. Viscoelastic materials have elements of both of these properties and, as such, exhibit time dependent strain. The viscoelastic polymer is generally not a liquid or granular substance at room temperature.

In one aspect, the viscoelastic polymer has the following properties: (1) a Shore 00 Durometer from 40 to 70, 42 to 65, or 45 to 55; (2) stress at 200% elongation (psi) (ASTMD-412) from 10 to 80, 12 to 75, or 15 to 60; (3) an ultimate tensile strength (psi) (ASTMD-412) from 10 to 150, 12 to 100, or 15 to 50; (4) a tear strength (pounds/inch) (ASTMD-624) from 3 to 50, 4 to 25, or 4 to 16; (5) elongation in percent (ASTMD-412) from 50 to 500, 75 to 400, or 100 to 350; (6) a storage modulus (psi/cycle at 72° F.) from 10 to 85, 15 to 45, or 10 to 20; and/or (7) a loss factor in percent from 50 to 99, 60 to 95, or 85 to 95. In another aspect, the viscoelastic polymer has a specific gravity greater than 1.00, from 1.05 to 3.00, 1.10 to 1.75, 1.25 to 1.50, 2.00 to 3.00, 2.50 to 3.00, or 2.60 to 2.80.

A variety of different viscoelastic polymers can be used herein. The viscoelastic polymers disclosed in U.S. Pat. No. 4,627,179, which are incorporated by reference, can be used herein. In one aspect the viscoelastic polymer may be a polyurethane or polyacrylamide. In another aspect, the viscoelastic polymer is a polyurethane sold under the tradename Akton® Viscoelastic Polymer manufactured by Action Products. In one aspect, the viscoelastic polymer is NEOPRENE (polychloroprene). In another aspect, the viscoelastic polymer is not a fluorocarbon or polyvinyl chloride.

The thickness of the viscoelastic polymer can vary depending upon the selection of the polymer, the article manufactured by the polymer, and the body part to be imaged. In one aspect, the article of clothing is made of a viscoelastic polymer with a thickness from 3/16″ to ¾′″, or from ¼″ to ⅝″.

The article composed of the viscoelastic polymer can be manufactured in a variety of different shapes and sizes. In one aspect, the article can be a wrap. The wrap may be any shape or size, with regards to length and width so that the wrap can be placed in intimate contact with that body part. In one aspect the wrap may have laces, Velcro, and the like to secure the wrap to the contours of the body part in a more fitting manner.

In another aspect, the article is a piece of clothing used to cover a body part. For example, the piece of clothing may cover the leg, arm, neck, elbow, knee, foot, hand or other body parts. In certain aspects, the piece of clothing can better accommodate (i.e., intimately contact) the contours of the body part. Like the wrap, the piece of clothing may have laces, Velcro, and the like to more securely fasten the article to the body part that is imaged.

In one aspect, the article is a slipper or a glove. In general, the slipper or glove is designed so that when placed on the foot or hand, respectively, a tight fit is produced. The slipper or glove may also have laces, Velcro, and the like to secure the slipper or glove, which places the foot or hand in intimate contact with the viscoelastic polymer in the slipper or glove.

The articles described herein can be manufactured by techniques known in the art. In one aspect the article of clothing is made from a single piece of material composed of viscoelastic polymer. In other aspects, the article of clothing is made from multiple pieces (i.e., two or more) of a viscoelastic polymer secured together by techniques known in the art. For example, the pieces of viscoelastic polymer may be attached to one another by sewing the pieces together or by melt bonding the pieces to one another. The manufacture of the article from multiple pieces makes it possible to produce the article in a variety of shapes and sizes. When attaching the pieces of material, it is desirable that the seams are on the outside of the article (i.e., not on the side that is in contact with the body part).

In certain aspects, the article consists essentially of the viscoelastic polymer. In this aspect, greater than 60%, greater than 70%, greater than 80%, greater than 90%, or greater than 95% by weight of the article is composed of viscoelastic polymer. For example, when the article is a glove, the glove can be entirely composed of viscoelastic polymer. Alternatively, when the article is a slipper, the slipper can optionally contain a sole. It is contemplated that the article nay include other features such as laces or Velcro to help fasten the article of clothing; however, such features are not in intimate contact with the body part.

The articles described herein are generally lightweight and easy to apply. In certain aspects, the article can be cleaned and reused, while in other aspects, the article can be disposed after use. The articles are generally inexpensive to produce relative to other articles currently used to increase fat saturation, which is another advantage of the present invention.

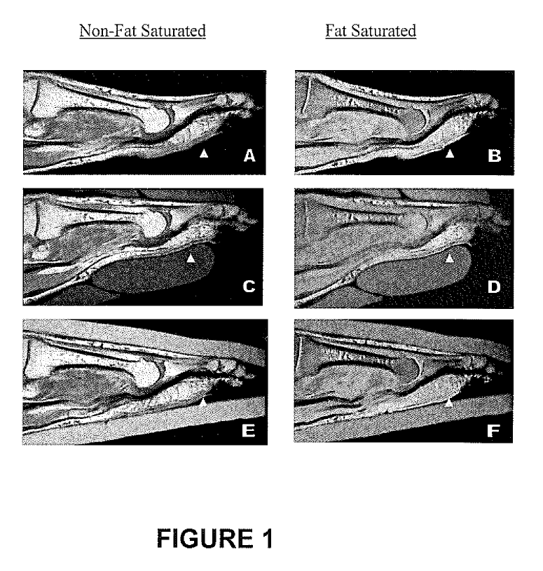

The articles and methods described herein are useful in improving MRI of body parts that are generally difficult to image. In one aspect, the article reduces the effect of air/tissue interfaces on fat saturation, thereby improving the visibility of bone marrow and subcutaneous tissues in the foot. In other aspects, the articles and methods described herein can improve the sensitivity and specificity of MRI for detecting conditions associated with diabetes (e.g., osteomyelitis, cellulitis) and tissue viability in patients. FIG. 1 shows how the articles and methods described herein can improve MRI imaging. Referring to FIG. 1, images A, C, and E show an MRI image of a foot without fat saturation, while the fat in images B, D, and F has been saturated. Standard techniques without augmentation are shown in A and B, the water bag technique is shown in C and D, and the use of the viscoelastic polymer (Akton®) is shown in E and F. Fat is indicated by bright (white) signal intensity on non-fat-saturated images (A, C, E) compared to decreased bright signal on fat-saturated images (B, D, F). Image F, which is produced with the aid of a viscoelastic polymer, reveals improved fat saturation compared to water bags (D) or standard technique (B). The white arrows demonstrate plantar fat where the differences are best visualized.

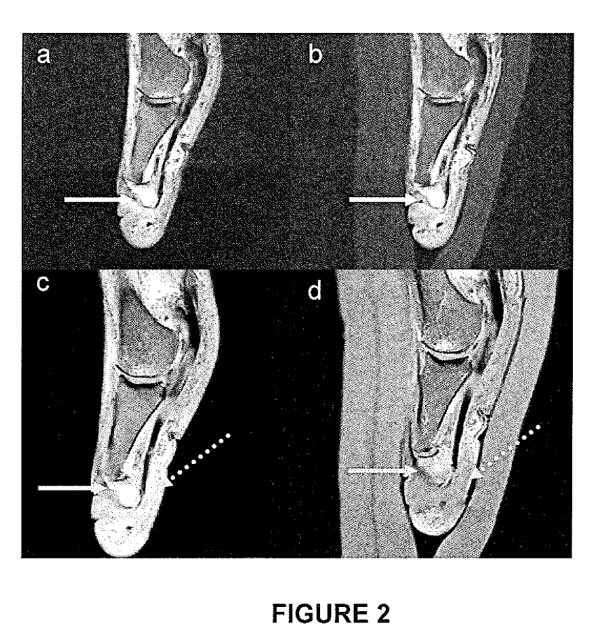

FIG. 2 shows osteomyelitis of the first toe, distal phalanx. Images a and c are without viscoelastopolymer, while images b and d are with viscoelastopolymer. Fluid sensitive images (T2 with fat saturation) (a and b) demonstrate increased fluid in the first toe, distal phalanx. With contrast and in the absence of viscoelastopolymer (c) the increased signal within the distal phalanx (solid white arrow) may be mistaken for artifact because of the overlying increased signal in the soft tissue (dotted white arrow), while with the viscoelastopolymer (d) the signal in the distal phalanx (solid white arrow) is unmistakably due to enhancement as there is no increased signal in the soft tissues (dotted white arrow).

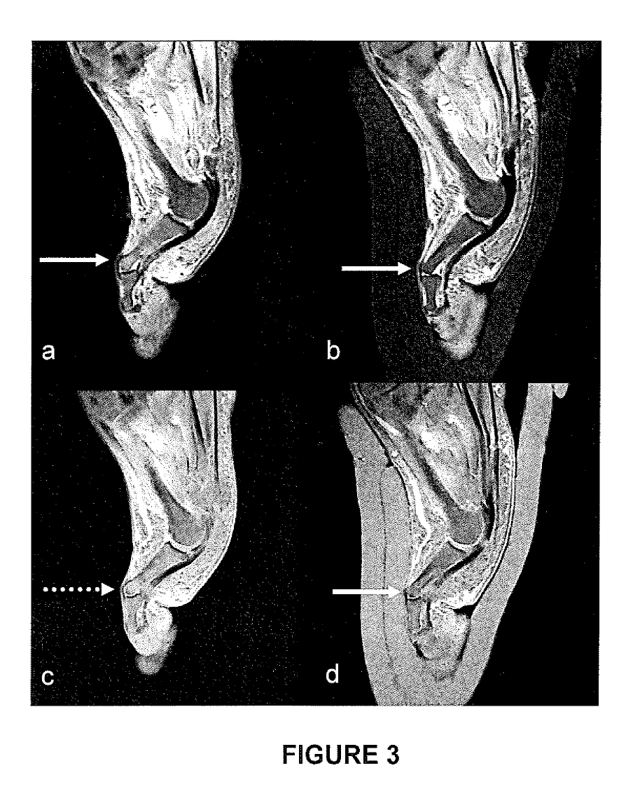

FIG. 3 shows an artifact in the second toe (head of proximal phalanx) that could be mistaken for osteomyelitis. Images a and c are without viscoelastopolymer, while images b and d are with viscoelastopolymer. Fluid sensitive images (T2 with fat saturation) (a and b) demonstrate questionable fluid in the head of the distal phalanx without viscoelastopolymer (arrow, a), which is not apparent with viscoelastopolymer (arrow, h). With contrast and in the absence of viscoelastopolymer (c) the increased signal within the head of the proximal phalanx (c, dotted arrow) may be mistaken for osteomyelitis, while with the viscoelastopolymer (d) no enhancement is visualized in the head of the proximal phalanx (solid arrow).

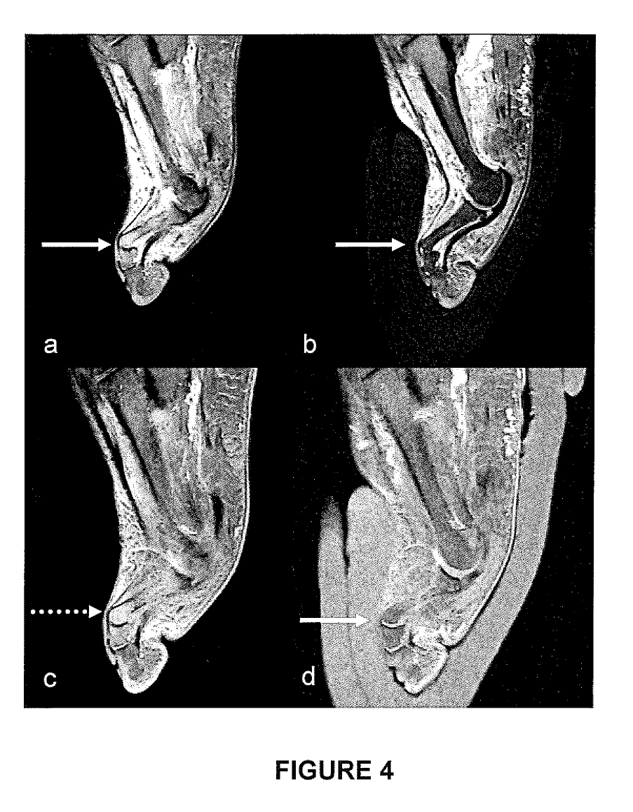

FIG. 4 shows an artifact in the third toe (head of proximal phalanx) that could be mistaken for osteomyelitis. Images a and c are without viscoelastopolymer, while images b and d are with viscoelastopolymer. Fluid sensitive images (T2 with fat saturation) (a and b) demonstrate questionable fluid in the head of the distal phalanx without viscoelastopolymer (arrow, a), which is not apparent with viscoelastopolymer (arrow, b). With contrast and in the absence of viscoelastopolymer (c) the increased signal within the head of the proximal phalanx (c, dotted arrow) may be mistaken for osteomyelitis, while with the viscoelastopolymer (d) there is no enhancement in the head of the proximal phalanx (solid arrow).

Although FIGS. 1-4 depict the imaging of a foot, the articles and methods described herein can improve visualization of pathology in other disease processes, such as arthritis, trauma, tumors and plan to extend the use of the material for imaging the shoulder, hip, elbow, wrist, hand, knee, ankle and leg.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for improving MRI imaging of a body part comprising (a) placing an article comprising a solid viscoelastic polymer on the surface of the body part wherein the article encompasses the body part, wherein the polymer is not polyvinyl chloride or a fluorocarbon, and the article is in intimate contact with the body part to be imaged, and (b) imaging the body part.

2. The method of claim 1, wherein the method increases fat saturation of the body part during imaging.

3. The method of claim 1, wherein the article comprises a wrap.

4. The method of claim 1, wherein the article comprises a piece of clothing.

5. The method of claim 1, wherein the article comprises a slipper or glove.

6. The method of claim 1, wherein the article consists essentially of a solid viscoelastic polymer.

7. The method of claim 1, wherein the article is a slipper or glove consisting essentially of a solid viscoelastic polymer.

8. The method of claim 1, wherein the viscoelastic polymer comprises polyurethane or polyacrylamide.

9. The method of claim 1, wherein the viscoelastic polymer has a thickness from 3/16″ to ¾′″.

10. The method of claim 1, wherein the viscoelastic polymer has a thickness from ¼″ to ⅝″.

11. The method of claim 1, wherein the body part comprises a foot, hand, knee, neck, ankle, or elbow.

12. The method of claim 1, wherein subject is afflicted with diabetes and the body part is the foot or hand.

13. An article of clothing for improving MRI imaging of a body part, wherein the article consists essentially of a viscoelastic polymer, and the article is in intimate contact with the body part when placed on the body part, and the polymer is not polyvinyl chloride.

14. The article of claim 13, wherein the article comprises a slipper or glove.

15. The article of claim 13, wherein the viscoelastic polymer comprises polyurethane or polyacrylamide.

16. The article of claim 13, wherein the viscoelastic polymer has a thickness from 3/16″ to ¾′″.

17. The article of claim 13, wherein the viscoelastic polymer has a thickness from ¼″ to ⅝″.

18. The article of claim 13, wherein the viscoelastic polymer is not a fluorocarbon.

19. The article of claim 13, wherein the article is disposable.

20. The article of claim 13, wherein the article comprises one piece of viscoelastic polymer.

21. The article of claim 13, wherein the article comprises two or more pieces of viscoelastic polymer attached to each other.
